(19) United States
(12) Patent Application Publication
(10) Pub. No.: US 2024/0010974 A1
(43) Pub. Date: Jan. 11, 2024

(54) METHOD OF PRODUCING A CELL POPULATION COMPRISING AN AMNION-DERIVED MESENCHYMAL STEM CELL

(71) Applicant: KANEKA CORPORATION, Osaka (JP)

(72) Inventors: Chiho KOBAYASHI, Kobe-shi (JP); Nobuhiko SATO, Kobe-shi (JP); Rio MASAYASU, Kobe-shi (JP)

(73) Assignee: KANEKA CORPORATION, Osaka (JP)

(21) Appl. No.: 18/372,374

(22) Filed: Sep. 25, 2023

Related U.S. Application Data
(63) Continuation of application No. PCT/JP2022/014149, filed on Mar. 24, 2022.

(30) Foreign Application Priority Data
Mar. 26, 2021 (JP) 2021-052792

(51) Int. Cl. C12N 5/0735

(52) U.S. Cl. CPC C12N 5/0606; C12N 2506/025; C12N 2501/734; C12N 2527/00

(57) ABSTRACT

An object of the present invention is to provide a method of producing a cell population comprising mesenchymal stem cells, comprising efficiently isolating a cell population comprising mesenchymal stem cells at high purity from an amnion. Provided is a method of producing a cell population comprising amnion-derived mesenchymal stem cells, comprising (1) storing an amnion in a medium for 4 hours or longer at −1° C. or higher and 25° C. or lower, followed by (2) isolating the amnion from the medium and treating the amnion with an enzyme; and (3) culturing a cell fraction comprising mesenchymal stem cells after the treatment with the enzyme.

### CROSS-REFERENCE TO RELATED APPLICATIONS

This application is a Continuation of PCT International Application No. PCT/JP2022/014149 filed on Mar. 24, 2022, which claims priority under 35 U.S.C. § 119 (a) to Japanese Patent Application No. 2021-052792, filed in Japan on Mar. 26, 2021, all of which are hereby expressly incorporated by reference into the present application.

### TECHNICAL FIELD

The present invention relates to a method of producing a cell population comprising an amnion-derived mesenchymal stem cells.

### BACKGROUND ART

Mesenchymal stem cells are somatic stem cells that are reported to be present in, for example, the bone marrow, fat tissue, and dental pulp. In recent years, mesenchymal stem cells are found to be present in fetal appendages, such as the placenta, umbilical cord, and fetal membrane. Mesenchymal stem cells have not only an ability to differentiate into bone, cartilage, and fat but also have an immunosuppressive ability. Thus, clinical applications are ongoing in the treatment of, e.g., acute graft versus host disease (GVHD) and Crohn's disease.

The amnion, which is a type of fetal appendages, is a tissue comprising many mesenchymal stem cells and thus has drawn attention as a promising cell source for mesenchymal stem cells. However, fetal appendages comprising an amnion comprise a large quantity of epithelial cells in addition to mesenchymal stem cells. This necessitates separation of mesenchymal stem cells from epithelial cells to obtain mesenchymal stem cells in high purity. According to conventional techniques, inclusion of epithelial cells was prevented to a certain extent by physical detaching unnecessary tissue pieces attached to the amnion (Patent Document 1; Non-Patent Documents 1 to 3). According to such techniques, however, it was difficult to remove epithelial cells existing inside the amnion. If epithelial cells are once included into culturing steps, it is very difficult to separate and isolate them because they are both adherent cells. In such circumstances, a method for reducing the inclusion of epithelial cells as small as possible is desired.

### CITATION LIST

### Patent Literature

Patent Literature 1: WO 2015/025810

### Non-Patent Literature

Non-Patent Literature 1: Am. J. Obstet. Gynecol., 2004; 190 (1): 87-92.

Non-Patent Literature 2: Am. J. Obstet. Gynecol., 2006; 194 (3): 664-73.

Non-Patent Literature 3: Current Protocols in Stem Cell Biology, 1E. 5

### SUMMARY OF INVENTION

### Technical Problem

An object of the present invention is to provide a method of producing a cell population comprising mesenchymal stem cells, and preferably comprising a very small amount of or no epithelial cells, comprising efficiently isolating a cell population comprising mesenchymal stem cells at high purity from an amnion.

### Solution to Problem

In the course of investigations for solving the above-described problem, the present inventors cut the amnion into small pieces and then carried out enzymatic treatment in order to improve the efficiency of the enzymatic treatment for isolating a cell population comprising mesenchymal stem cells from the amnion. As a result, the present inventors found that the ratio of the epithelial cells included in the cell population comprising the mesenchymal stem cells of interest was significantly increased. The present inventors have further conducted extensive studies, and as a result, surprisingly found that the proportion of the included epithelial cells could be reduced and the cell population comprising mesenchymal stem cells at high purity could be efficiently isolated from the amnion by soaking the amnion in a medium, storing the amnion for a certain period of time, and then carrying out enzymatic treatment, wherein the enzymatic treatment is preferably carried out before culturing the cells.

Based on the findings described above, the present invention described below has been completed.

In a one embodiment, the present invention relates to a method of producing a cell population comprising amnion-derived mesenchymal stem cells, comprising
(1) a step of storing an amnion in a medium for 4 hours or longer at −1° C. or higher and or lower;
(2) a step of treating the amnion with an enzyme thereafter; and
(3) a step of culturing a cell fraction comprising mesenchymal stem cells after the treatment with the enzyme.

The present invention additionally provides the following.

[1] A method of producing a cell population comprising an amnion-derived mesenchymal stem cell, comprising
(1) a step of storing an amnion in a medium for 4 hours or longer at −1° C. or higher and or lower;
(2) a step of treating the amnion with one or more enzymes thereafter; and
(3) a step of culturing a cell fraction comprising mesenchymal stem cells after the treatment with the one or more enzymes.

[2] The method of producing a cell population comprising mesenchymal stem cells according to [1], further comprising isolating the amnion from the medium before the treatment with the one or more enzymes.

[3 ] The method of producing a cell population comprising mesenchymal stem cells according to [1] or [2], wherein the medium is an aqueous solution, a gel, or a sol.

[4] The method of producing a cell population comprising mesenchymal stem cells according to any one of [1] to [3], wherein the one or more enzymes comprise at least one selected from the group consisting of trypsin, collagenase, and dispase.

[5 ] The method of producing a cell population comprising mesenchymal stem cells according to any one of [1] to [4], wherein the amnion stored in the medium in the step (1) is shredded.

[6] The method of producing a cell population comprising mesenchymal stem cells according to any one of [1] to [5], wherein the amnion is stored at −1° C. or higher and 10° C. or lower in the step (1).

In another embodiment, the present invention provides the following.

[1A] A method of producing a cell population comprising amnion-derived mesenchymal stem cells, comprising
(1) a step of storing an amnion in a medium for 4 hours or longer at −1° C. or higher and 25° C. or lower;
(2) a step of treating the amnion with one or more enzymes thereafter, to obtain an enzyme treated solution;
(3) a step of removing an undigested amnion fraction from said enzyme treated solution to obtain an enzyme digested fraction having a reduced proportion of epithelial cells and comprising amnion-derived mesenchymal stem cells; and
(4) a step of culturing said enzyme digested amnion fraction comprising mesenchymal stem cells after the treatment with the one or more enzymes.

[2A] The method of producing a cell population comprising mesenchymal stem cells according to [1A], further comprising isolating the amnion from the medium before the treatment with the one or more enzymes.

[3A] The method of producing a cell population comprising mesenchymal stem cells according to [1A] or [2A], wherein the medium is an aqueous solution, a gel, or a sol.

[4A] The method of producing a cell population comprising mesenchymal stem cells according to any one of [1A] to [3A], wherein the one or more enzymes comprise at least one selected from the group consisting of trypsin, collagenase, and dispase.

[5A] The method of producing a cell population comprising mesenchymal stem cells according to any one of [1A] to [4A], wherein the amnion stored in the medium in the step (1) is shredded.

[6A] The method of producing a cell population comprising mesenchymal stem cells according to any one of [1A] to [5A], wherein the amnion is stored at −1° C. or higher and 10° C. or lower in the step (1).

The present description encompasses the disclosure of Japanese Patent Application No. 2021-052792, which forms the basis of the priority of the present application. In this connection, FIG. 1-1 and FIG. 1-2 in the present description correspond to FIG. 1 and Table 1 in Japanese Patent Application No. 2021-052792. FIG. 2-1 and FIG. 2-2 in the present description correspond to FIG. 2 and Table 2 in Japanese Patent Application No. 2021-052792. FIG. 3-1 and FIG. 3-2 in the present description correspond to FIG. 3 and Table 3 in Japanese Patent Application No. 2021-052792. FIG. 4-1 and FIG. 4-2 in the present description correspond to FIG. 4 and Table 4 in Japanese Patent Application No. 2021-052792. FIG. 5-1 and FIG. 5-2 in the present description correspond to FIG. 5 and Table 5 in Japanese Patent Application No. 2021-052792. FIG. 6-1 and FIG. 6-2 in the present description correspond to FIG. 6 and Table 6 in Japanese Patent Application No. 2021-052792.

### Advantageous Effects of Invention

According to the present invention, a cell population comprising mesenchymal stem cells at high purity can be efficiently isolated from the amnion. The cell population comprising mesenchymal stem cells at high purity thus obtained has a very low number of epithelial cells (a low percentage of epithelial cells) or no epithelial cells. Thus, a cell preparation (a pharmaceutical composition) can be produced at low cost.

### BRIEF DESCRIPTION OF DRAWINGS

FIG. 1-1 shows the microscopic observation image of the cell population at passage 0 derived from Donor A in Comparative Example 1.

FIG. 1-2 shows the proportions of cells positive for surface antigens CD73, CD90, and CD326 in the cell population at passage 0 derived from Donor A in Comparative Example 1.

FIG. 2-1 shows the microscopic observation image of the cell population at passage 0 derived from Donor A in Example 1 (left: the cell population obtained from the amnion after storage for 120 hours in Process 1; right: the cell population obtained from the amnion after storage for 216 hours in Process 1).

FIG. 2-2 shows the proportions of cells positive for surface antigens CD73, CD90, and CD326 in the cell population at passage 0 derived from Donor A in Example 1 after storage for 120 hours or 216 hours.

FIG. 3-1 shows the microscopic observation image of the cell population at passage 0 derived from Donor A in Example 2 (left: the cell population obtained from the amnion after storage for 120 hours in Process 1; right: the cell population obtained from the amnion after storage for 216 hours in Process 1).

FIG. 3-2 shows the proportions of cells positive for surface antigens CD73, CD90, and CD326 in the cell population at passage 0 derived from Donor A in Example 2 after storage for 120 hours or 216 hours.

FIG. 4-1 shows the microscopic observation image of the cell population at passage 0 derived from Donor B in Example 3.

FIG. 4-2 shows the proportions of cells positive for surface antigens CD73, CD90, and CD326 in the cell population at passage 0 derived from Donor B in Example 3.

FIG. 5-1 shows the microscopic observation image of the cell population at passage 0 derived from Donor A in Example 4.

FIG. 5-2 shows the proportions of cells positive for surface antigens CD73, CD90, and CD326 in the cell population at passage 0 derived from Donor A in Example 4.

FIG. 6-1 shows the microscopic observation image of the cell population at passage 0 derived from Donor C in Example 5.

FIG. 6-2 shows the proportions of cells positive for surface antigens CD73, CD90, and CD326 in the cell population at passage 0 derived from Donor C in Example 5.

FIG. 7-1 shows the microscopic observation image of the cell population at passage 0 derived from Donor B in Example 6 (left: the cell population obtained from the amnion after storage for 48 hours in Process 1; right: the cell population obtained from the amnion after storage for 144 hours in Process 1).

FIG. 7-2 shows the proportions of cells positive for surface antigens CD73, CD90, and CD326 in the cell population at passage 0 derived from Donor B in Example 6 after storage for 48 hours or 144 hours.

FIG. 8-1 shows the microscopic observation image of the cell population at passage 0 derived from Donor B in Example 7 (left: the cell population obtained from the amnion after storage for 48 hours in Process 1; right: the cell population obtained from the amnion after storage for 144 hours in Process 1).

FIG. 8-2 shows the proportions of cells positive for surface antigens CD73, CD90, and CD326 in the cell population at passage 0 derived from Donor B in Example 7 after storage for 48 hours or 144 hours.

FIG. 9-1 shows the microscopic observation image of the cell population at passage 0 derived from Donor B in Example 8 (left: the cell population obtained from the amnion after storage for 48 hours in Process 1; right: the cell population obtained from the amnion after storage for 144 hours in Process 1).

FIG. 9-2 shows the proportions of cells positive for surface antigens CD73, CD90, and CD326 in the cell population at passage 0 derived from Donor B in Example 8 after storage for 48 hours or 144 hours.

FIG. 10-1 shows the microscopic observation image of the cell population at passage 0 derived from Donor C in Example 9 (left: the cell population obtained from the amnion after storage for 4 hours in Process 1; right: the cell population obtained from the amnion after storage for 94 hours in Process 1).

FIG. 10-2 shows the proportions of cells positive for surface antigens CD73, CD90, and CD326 in the cell population at passage 0 derived from Donor C in Example 9 after storage for 4 hours or 94 hours.

FIG. 11-1 shows the microscopic observation image of the cell population at passage 0 derived from Donor C in Example 10 (left: the cell population obtained from the amnion after storage for 4 hours in Process 1; right: the cell population obtained from the amnion after storage for 94 hours in Process 1).

FIG. 11-2 shows the proportions of cells positive for surface antigens CD73, CD90, and CD326 in the cell population at passage 0 derived from Donor C in Example 10 after storage for 4 hours or 94 hours.

FIG. 12-1 shows the microscopic observation image of the cell population at passage 0 derived from Donor C in Example 10. In FIG. 12-1, regions inside the circles indicate epithelial cells.

FIG. 12-2 shows the proportions of cells positive for surface antigens CD73, CD90, and CD326 in the cell population at passage 0 derived from Donor C in Comparative Example 2 after storage for 4 hours.

FIG. 13 shows the microscopic observation image of the cell population at passage 0 derived from Donor C in Comparative Example 3 (left: the cell population 3 days after seeding in Process 4; right: the cell population 7 days after seeding in Process 4).

### DESCRIPTION OF EMBODIMENTS

Hereafter, embodiments of the present invention are described in detail. The following description is provided to facilitate understanding of the present invention, and the scope of the present invention is not limited to the following embodiments. Other embodiments in which the configuration of the embodiments described below is substituted as appropriate by a person skilled in the art are also comprised in the scope of the present invention.

### [1] Description of Terms

The term “fetal appendage” used herein refers to a fetal membrane, a placenta, an umbilical cord, and an amnionic fluid. The term “fetal membrane” refers to a fetal sac comprising fetal amnionic fluid, and a fetal membrane consists of an amnion, a chorionic membrane, and a decidual membrane from the inside. The amnion and the chorionic membrane are from a fetus in their origin. The term “amnion” refers to a transparent, thin, and poorly vascularized membrane in the innermost layer of the fetal membrane. The inner layer of the amnion (which is also referred to as an “epithelial cell layer”) is covered by a layer of epithelial cells having an ability of secretion and secretes amnionic fluid. The outer layer of the amnion (which is also referred to as an “extracellular matrix layer” and corresponds to the stroma) comprises mesenchymal stem cells.

The term “mesenchymal stem cell (MSC)” or “mesenchymal stem cells (MSCs)” used herein refers to a stem cell or cells which satisfies the definition provided below, and the term is used interchangeably with the term “mesenchymal stromal cell.” The term “mesenchymal stem cell” may be referred to as “MSC” or “MSCs” herein.

### Definition of a Mesenchymal Stem Cell

i) It shows adhesiveness to plastics under culture conditions in a standard medium. A standard medium is a medium which is a basal medium (e.g., αMEM medium) supplemented with serum, a serum substitute reagent, or a growth factor (e.g., a human platelet lysate which is a serum substitute reagent).

ii) It is positive for surface antigens CD105, CD73, and CD90 and negative for CD45.

With respect to “a cell population comprising mesenchymal stem cells”, the form thereof is not particularly limited herein. Examples thereof include a cell pellet, a cell aggregate, a cell float, and a cell suspension.

The term “amnion-derived mesenchymal stem cells” used herein refers to mesenchymal stem cells derived from an amnion, and the term may be referred to as “amnion-derived MSCs.”

In the present invention, a “medium” is not particularly limited, and a medium may have any condition, property, or structure. For example, a medium may be in any form, such as a solid, liquid, or gas, or a mixture of them. Specific examples include a gel, a sol, and an aqueous solution. A gel refers to colloidal particles which are dispersed in a liquid or gas and have lost fluidity. Examples thereof include Amorphophallus konjac, Yokan (adzuki bean jelly), agar, and pudding. A sol refers to colloidal particles which are dispersed in a liquid or gas and have not lost fluidity. Examples thereof comprise milk, yogurt, and oil. As a sol/gel, a colloid whose dispersion medium is water is preferable, and a so-called hydrogel is more preferable. An aqueous solution refers to, e.g., a buffer, an isotonic liquid, a hypotonic liquid, or a hypertonic liquid. A buffer or isotonic liquid is more preferable so as to reduce damages on tissues. Examples thereof include a buffer such as phosphate-buffered saline (PBS), a balanced salt solution such as Hanks' balanced salt solution (HBSS) and Earle's balanced salt solution (EBSS), an infusion solution such as Ringer's solution, lactated Ringer's solution, and physiologic saline, a culture solution, an albumin solution, an aqueous solution comprising a blood-derived component, and a mixture of them. The medium may be supplemented with an antibiotics so as to suppress bacterial proliferation.

From the viewpoint of biocompatibility, the medium used in the present invention may comprise at least one selected from the group consisting of a protein, a peptide, a polysaccharide, and a synthetic polymer. As the protein described above, gelatin, collagen, fibrin, soybean protein, or the like can be used. As a polysaccharide or a substance comprising a polysaccharide, agarose, pectin, carrageenan, curdlan, chitin, chitosan, alginic acids, soybean polysaccharides, celluloses such as carboxymethyl cellulose, mannans, gum Arabic, gellan gum, guar gum, xanthan gum, starch, agar, fucoidan, and the like can be used. As a synthetic polymer, e.g., a synthetic peptide (a self-assembling peptide, such as PanaceaGel and PuraMatrix), polyvinyl alcohol, propylene glycol, silicon, and polyacrylamide can be used. Any of these may be used alone or in combinations of two or more. It is preferable that the “medium” in the present invention does not contain a component which would affect an amnion tissue, for example, an enzyme, such as trypsin, collagenase, or dispase.

The “culture solution” used herein is not particularly limited, and can be prepared by supplementing as necessary a basal medium; i.e., any liquid medium for cell culture, with other components (e.g., albumin, a blood-derived component, and a growth factor), as appropriate.

Examples of the basal medium described above that can be used include, but are not particularly limited to, BME medium, BGJb medium, CMRL1066 medium, Glasgow MEM medium, Improved MEM Zinc Option medium, IMDM medium (Iscove's Modified Dulbecco's Medium), Medium 199 medium, Eagle MEM medium, αMEM (Alpha modification of Minimum Essential Medium Eagle) medium, MEM-α (Minimum Essential Medium α) medium, DMEM medium (Dulbecco's Modified Eagle's Medium), Ham's F10 medium, Ham's F12 medium, RPMI 1640 medium, Fischer's medium, and a mixture of them (e.g., DMEM/F12 medium (Dulbecco's Modified Eagle's Medium/Nutrient Mixture F-12 Ham)). Various commercially available serum-free media can also be used.

Examples of other components to be added to said basal medium include albumin, a blood-derived component, and a growth factor. In an aspect in which albumin is added to said basal medium, the albumin concentration is preferably 0.05% or more by mass to 5% or less by mass. Examples of blood-derived components comprise various sera (e.g., animal-derived serum, such as fetal bovine serum (FBC or FCS), human serum, and serum prepared from a platelet-rich plasma or platelet lysate derived from the blood of various animals and/or a human), platelet lysates derived from the blood of various animals and/or a human, and plasma. Human serum may be derived from an individual who is identical to the individual from which tissue comprising an adherent cell was obtained or it may be derived from a different individual. In an aspect in which a blood-derived component is added to said basal medium, the concentration of the blood-derived component is preferably 2% or more by volume and 40% or less by volume, and more preferably 3% or more by volume to 30% or less by volume. In an aspect in which a growth factor is added, a reagent to stabilize the growth factor in a medium (e.g., an anticoagulant such as heparin, a gel, or polysaccharides) may further be added, in addition to the growth factor. Alternatively, the growth factor that is stabilized in advance may be added to said basal medium. Examples of growth factors that can be used include, but are not particularly limited to, fibroblast growth factor (FGF), epithelial cell growth factor (EGF), transforming growth factor (TGF), vascular endothelial cell growth factor (VEGF), platelet-derived growth factor (PDGF), and a family of them.

### [2] A Method of Producing a Cell Population Comprising an Amnion-derived Mesenchymal Stem Cell

A step of sampling fetal appendages comprising mesenchymal stem cells can be performed in a manner described below in the case of, for example, an amnion tissue. At the time of birth, fetal appendages, such as a placenta and a fetal membrane, are sampled, and the amnion is detached from the stump end of the fetal membrane.

Thereafter, the sampled amnion is soaked and stored in the “medium” described in [1] Description of terms. The amnion stored in the medium is preferably an amnion which is shredded (cut into small pieces) so as to improve the efficiency for subsequent enzymatic treatment. Alternatively, the sampled amnion may be directly soaked and then shredded.

A duration of amnion storage is required to be “4 hours or longer.” For example, such duration is 4 hours or longer or 5 hours or longer, preferably 6 hours or longer, 8 hours or longer, or 10 hours or longer, and more preferably 12 hours or longer, 24 hours or longer, or 48 hours or longer. While the upper limit of the duration of amnion storage is not particularly limited, the duration may be, for example, within 30 days, 25 days, 20 days, 15 days, or 10 days, and preferably within 9 days, 7 days, 5 days, or 3 days. Examples of the storage duration include 4 hours and longer and within 30 days, 5 hours or longer and within 20 days, 6 hours or longer and within 10 days, 12 hours and longer and within 9 hours, or 24 hours or longer and within 7 days.

In the present invention, the temperature for amnion storage is required to satisfy “−1° C. or higher and 25° C. or lower.” The upper limit of storage temperature is not particularly limited, provided that it is 25° C. or lower. For example, the upper limit may be 20° C., 19° C., 18° C., 17° C., 16° C., 15° C., 14° C., 13° C., 12° C., 11° C., 10° C., 9° C., or 8° C. or lower. The lower limit of storage temperature is not particularly limited, provided that water in the medium is not frozen. For example, the lower limit is −1° C., 0° C., 1° C., 2° C., 3° C., 4° C., 5° C., 6° C., or 7° C. or higher. Examples of a temperature range include −1° C. or higher and 20° C. or lower, −1° C. or higher and 15° C. or lower, or −1° C. or higher and 10° C. or lower.

A step of isolating a cell fraction comprising mesenchymal stem cells from the tissue after storage can be performed, for example, in a manner described below. The tissue after storage described above may be directly subjected to an enzymatic treatment. Preferably, the tissue is isolated from the medium, subjected to enzymatic treatment, then centrifuged to separate adherent cells, and repeatedly subjected to washing with a wash solution and centrifugation a plurality of times, so as to obtain a cell fraction comprising a mesenchymal stem cell. In such a case, the amnion may be shredded (cut into small pieces) with scissors before the enzymatic treatment to improve the efficiency for digestion with an enzyme. Alternatively, the amnion may be washed, according to need. An enzyme is not limited, provided that it is a digestive enzyme that can at least partially digest the amnion tissue and separate at least some adherent cells contained in the amnion tissue. A digestive enzyme may be, for example, a protease. An example of a solution that can be used for the enzymatic treatment in the present invention is, but is not limited to, an enzyme liquid comprising one or more enzymes selected from e.g., trypsin, collagenase, dispase, and the like. A solution used for the enzymatic treatment can comprise a component, such as magnesium salt or calcium salt, which is necessary for the enzymatic treatment.

A step of producing a cell population comprising a mesenchymal cell from a cell fraction comprising mesenchymal stem cells isolated from the tissue comprising an adherent cell can be performed, for example, in a manner described below. Initially, a cell suspension which is the cell fraction comprising mesenchymal stem cells described above, is centrifuged, a supernatant is removed, and the resulting cell pellet is suspended in a medium. Subsequently, cells are seeded in a culture vessel and cultured in the presence of 3% to 5% CO₂ at 37° C. in a medium to become 95% or lower confluence. As the medium described above, for example, the “culture solution” described in [1] Description of terms can be used, although the medium is not limited thereto in the present invention. The cells obtained by the culture as described above are cells that are obtained by one-time culture.

A duration of the one-time culture described above can be, for example 2 to 21 days, and such duration is more preferably 3 to 19 days, and further preferably 4 to 17 days.

The cells obtained by one-time culture can further be passaged and cultured, for example, in a manner described below. Initially, the cells obtained by one-time culture are released from a culture vessel by the cell releasing means described below. Subsequently, the resulting cell suspension is centrifuged, a supernatant is removed, and the resulting cell pellet is suspended in a medium. Finally, cells are seeded in a culture vessel and cultured in the presence of 3% to 5% CO₂ at 37° C. in a medium to become 95% or lower confluence. As the medium, the “culture solution” described in [1] Description of terms can be used, although the medium is not limited thereto in the present invention.

The cells obtained by culture may be repeatedly passaged and cultured, so that the cells that have been passaged the “n” number of times can be obtained (“n” is an integer of 1 or larger). The lower limit of the passage number “n” is, for example, 1 or more, preferably 2 or more, more preferably 3 or more, and further preferably 4 or more, so as to produce a large number of cells. Further, the upper limit of the passage number “n” is preferably, for example, 25 or lower, 20 or lower, 15 or lower, or 10 or lower, so as not to permit senescence of the cell.

As the cell releasing means described above, for example, an agent for releasing (detaching) a cell may be used. Examples of an agent for releasing a cell which can be used include, but are not particularly limited to, trypsin, collagenase, dispase, and ethylenediaminetetraacetic acid (EDTA). A commercially available agent for releasing a cell may be used. Examples include, but are not limited to, a trypsin-EDTA solution (Thermo Fisher Scientific), TrypLE Select (Thermo Fisher Scientific), Accutase (Stemcell Technologies), and Accumax (Stemcell Technologies). A physical cell releasing means may be used. For example, a cell scraper (Corning) can be used, without limitations thereto. A single type of a cell releasing means may be used alone, or a plurality of types of cell releasing means may be used in combination.

In the present invention, the cell population comprising mesenchymal stem cells obtained as described above can also be cryopreserved. A means for cryopreserving the cell population comprising mesenchymal stem cells is not particularly limited. For example, the cell population can be stored in a program freezer, a deep freezer, or liquid nitrogen. When a program freezer is used, freezing temperature is preferably −30° C. or lower, −40° C. or lower, −50° C. or lower, −80° C. or lower, −90° C. or lower, −100° C. or lower, −150° C. or lower, −180° C. or lower, or −196° C. (liquid nitrogen temperature) or lower. When a program freezer is used, a freezing rate for freezing is preferably, for example, 15° C./min or lower, 11° C./min or lower, 10° C./min or lower, 9° C./min or lower, 5° C./min or lower, 2° C./min or lower, or 1° C./min or lower. When a program freezer is used as the means for freezing, for example, a freezing rate is adjusted to 1° C./min to 2° C./min at least when temperature is lowered from room temperature to −10° C., and otherwise a cooling rate may be changed as appropriate to reach the target freezing temperature (e.g., −80° C. to −150° C.) in the end. When soaking in liquid nitrogen is used as the freezing means described above, for example, temperature can be rapidly lowered to −196° C. to freeze followed by cryopreservation in liquid nitrogen (gas phase). Alternatively, the cell population can also be stored in liquid nitrogen (liquid phase).

When the cell population is to be frozen by the freezing means described above, the cell population described above may be frozen in any storage container. Examples of the storage containers include, but are not limited to, a cryotube, a cryovial, a freezing bag, and an infusion bag.

When the cell population is to be frozen by the freezing means described above, the cell population described above may be frozen in any cryopreservation solution. As the cryopreservation solution described above, a commercially available cryopreservation solution may be used. Examples include, but are not limited to, CP-1® (Kyokuto Pharmaceutical Industrial Co., Ltd.), BAMBANKER (LYMPHOTEC Inc), STEM-CELLBANKER (Nippon Zenyaku Kogyo Co., Ltd.), ReproCryo RM (ReproCELL, Inc.), CryoNovo (Akron Biotechnology), MSC Freezing Solution (Biological Industries), and CryoStor (HemaCare). A cryopreservation solution may be used alone, or a plurality of types of cryopreservation solutions may be used in combination.

The cryopreservation solution described above can comprise polysaccharides at a certain concentration. Preferable concentration of polysaccharides is, for example, 1% by mass or higher, 2% by mass or higher, 4% by mass or higher, or 6% by mass or higher. Preferable concentration of polysaccharides is, for example, 20% by mass or lower, 18% by mass or lower, 16% by mass or lower, 14% by mass or lower, or 13% by mass or lower. Examples of polysaccharides include, but are not limited to, hydroxylethyl starch (HES) and dextran (e.g., Dextran 40). Polysaccharide may be used alone, or a plurality of types of polysaccharides may be used in combination.

The cryopreservation solution described above can contain dimethyl sulfoxide (DMSO) at a certain concentration. Preferable concentration of DMSO is, for example, 1% by mass or higher, 2% by mass or higher, 3% by mass or higher, 4% by mass or higher, or 5% by mass or higher. Additionally, preferable concentration of DMSO is, for example, 20% by mass or lower, 18% by mass or lower, 16% by mass or lower, 14% by mass or lower, 12% by mass or lower, or 10% by mass or lower.

The cryopreservation solution described above may contain albumin at a certain concentration, which is higher than 0% by mass. Preferable concentration of albumin is, for example, 1% by mass or higher, 2% by mass or higher, 3% by mass or higher, or 4% by mass or higher. Preferable concentration of albumin is, for example, 30% by mass or lower, 20% by mass or lower, 10% by mass or lower, or 9% by mass or lower. Examples of albumin include, but are not limited to, bovine serum albumin (BSA), mouse albumin, and human albumin.

In an aspect of the present invention, the cell population comprising mesenchymal stem cells obtained by the production method to the present invention may satisfy that the proportions of CD73-positive, CD90-positive, and CD105-positive mesenchymal stem cells are each 80% or more.

CD73 means Cluster of Differentiation 73 and is a protein known also as 5-Nucleotidase or Ecto-5′-nucleotidase.

CD90 means Cluster of Differentiation 90 and is a protein known also as Thy-1.

CD105 means Cluster of Differentiation 105 and is a protein known also as Endoglin.

In the cell population obtained by the production method according to the present invention, the proportion of CD73-positive mesenchymal stem cells may be 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or 100%.

In the cell population obtained by the production method according to the present invention, the proportion of CD90-positive mesenchymal stem cells may be 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or 100%.

In the cell population obtained by the production method according to the present invention, the proportion of CD105-positive mesenchymal stem cells may be 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or 100%.

In an aspect of the present invention, the cell population comprising mesenchymal stem cells obtained by the production method according to the present invention may satisfy that the proportions of CD45-negative and CD31-negative mesenchymal stem cells are each 80% or more.

CD45 means Cluster of Differentiation 45 and is a protein known also as PTPRC (protein tyrosine phosphatase, receptor type, C) or LCA (leukocyte common antigen).

CD31 means Cluster of Differentiation 31 and is a protein known also as Hematopoietic progenitor cell antigen CD31.

In the cell population obtained by the production method according to the present invention, the proportion of CD45-negative mesenchymal stem cells may be 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or 100%.

In the cell population obtained by the production method according to the present invention, the proportion of CD31-negative mesenchymal stem cells may be 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or 100%.

Additionally, in the method of production according to the present invention, inclusion of epithelial cells can be reduced. Accordingly, the cell population obtained by the production method according to the present invention is characterized by a low proportion of cells positive for CD326, which is an epithelial cell marker. In the cell population obtained by the production method according to the present invention, specifically, the proportion of CD326-positive cells may be more preferably 10% or less (the negative rate may be 90% or more), 5% or less (the negative rate may be 95% or more), 4% or less (the negative rate may be 96% or more), 2% or less (the negative rate may be 98% or more), 1% or less (the negative rate may be 99% or more), or 0% (the negative rate may be 100%).

In the following examples, the present invention is described in detail, although the present invention is not limited to these examples.

### EXAMPLES

### Comparative Example 1: Examination of Amnion Storage

Process 1: Amnion Sampling

From a pregnant woman of an elective cesarean section case from which the written informed consent was obtained (Donor A), the fetal membrane and the placenta which are fetal appendages were sampled aseptically. The fetal membrane and the placenta obtained were accommodated in a vessel containing physiological saline, and the amnion was isolated from the stump end of the fetal membrane. The amnion was washed using a Hanks' balanced salt solution (without Ca and Mg), the weight of the sampled amnion was measured, and Process 2 was performed immediately with the use of about 1 g of the amnion.

Process 2: Enzymatic Treatment of Amnion and Acquisition of Amnion-derived MSCs

The amnion (about 1 g) was agitated with shaking in a Hanks' balanced salt solution (with Ca and Mg) comprising 240 PU/mL collagenase and 200 PU/mL dispase I at 37° C. for 90 minutes and the amnion was thus subjected to enzymatic treatment. After the enzymatic treatment, the solution was filtered through a strainer to remove an undigested amnion fraction, and a cell fraction comprising amnion-derived MSCs was obtained.

Process 3: Culture of Amnion-derived MSCs

The cell fraction comprising amnion-derived MSCs obtained in “Process 2: Enzymatic treatment of amnion and acquisition of amnion-derived MSCs” above was seeded in a culture vessel (T-25 flask, Corning) in an amount of ⅕ thereof and subjected to adherent culture in MEM-α (Minimum essential medium α) comprising 5% by volume (final concentration) of a human-blood-derived human platelet lysate (hPL). The cells subjected to adherent culture are referred to as the “cell population at passage 0.” When the cells were cultured to a subconfluent state, the cells were detached with the use of TrypLE Select, diluted using physiological saline comprising 2% by volume of hPL, and collected by centrifugation. The collected cell population was suspended in a cryopreservation solution comprising CP-1® (Kyokuto Pharmaceutical Industrial Co., Ltd.), 25% by mass human serum albumin, and physiological saline mixed at the proportion of 2:1:3, the cell suspension was slowly frozen to −80° C., and was thereafter cryopreserved at −80° C.

Process 4: Microscopic Observation of Cell Population at Passage 0 Comprising Cultured Amnion-derived MSCs

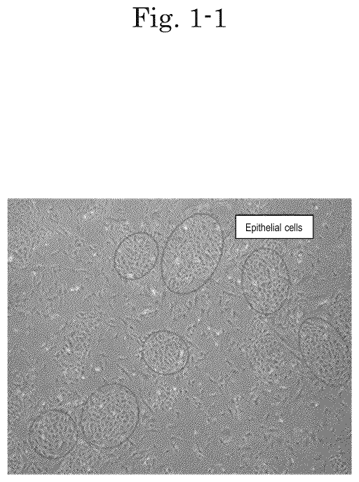

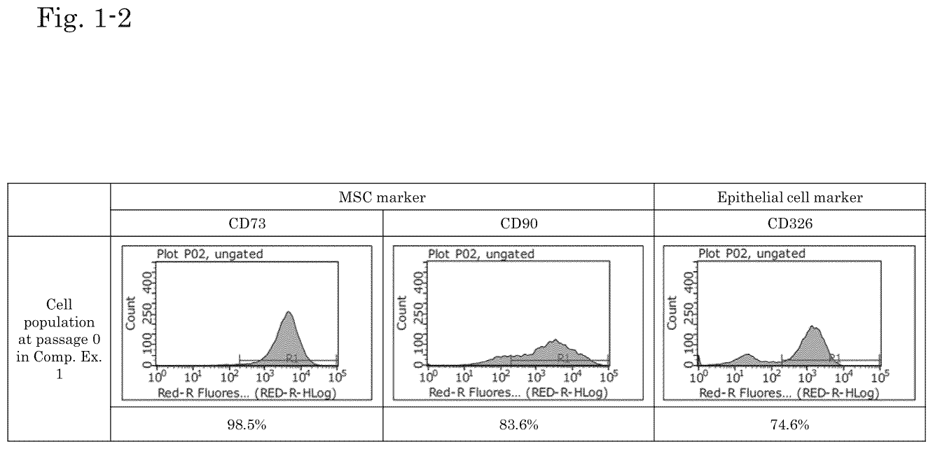

Concerning the cell population at passage 0 cultured in “Process 3: Culture of amnion-derived MSCs” described above, cell morphology was observed under an inverted culture microscope at any timing. The results are shown in FIG. 1-1.

The results demonstrate that, in addition to amnion-derived MSCs, a large number of epithelial cells (circles in FIG. 1-1) are included in the cell population at passage 0 derived from Donor A in Comparative Example 1.

Process 5: Surface Antigen Analysis

The cell population at passage 0 derived from Donor A in Comparative Example 1 was subjected surface antigen analysis (the CD73 positive rate, the CD326 positive rate, and the CD326 positive rate) using a flow cytometer.

Surface antigen analysis was performed using Guava easyCyte Single (MERCK) by subjecting 10,000 cells to analysis at the medium flow rate. In this analysis, APC REA control (S) (model no. 130-113-434, Miltenyi Biotec) was used as the isotype control antibody, APC Mouse Anti-Human CD73 (model no. 130-112-061, Miltenyi Biotec) was used as the anti-CD73 antigen antibody, APC Mouse Anti-Human CD90 (model no. 130-114-903, Miltenyi Biotec) was used as the anti-CD90 antigen antibody, and APC Mouse Anti-Human CD326 (model no. 130-111-117, Miltenyi Biotec) was used as the anti-CD326 antigen antibody.

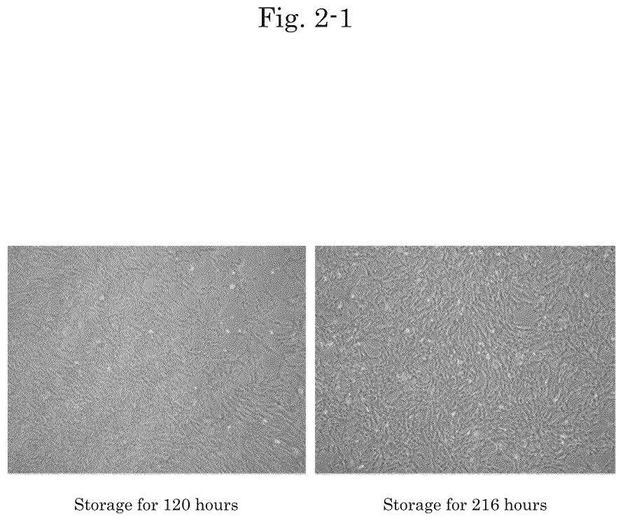

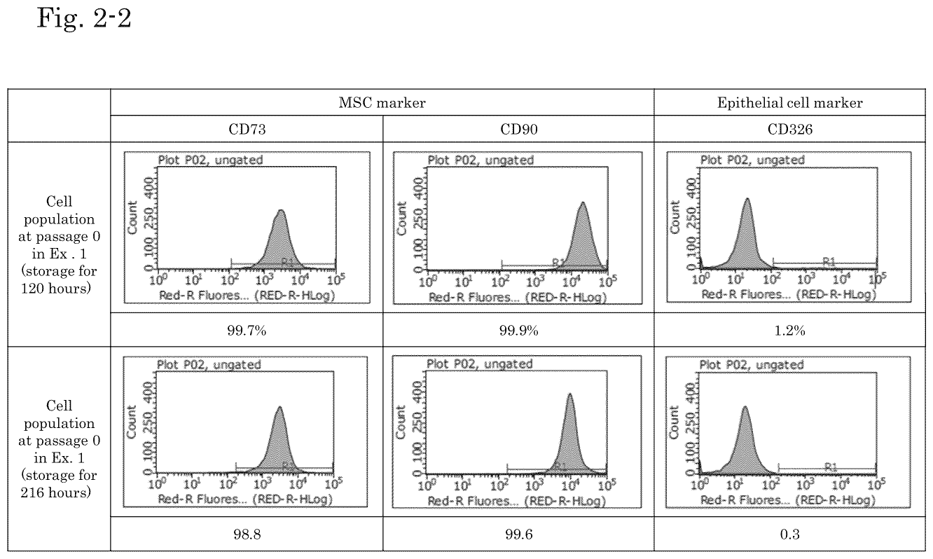

The results of surface antigen analysis are shown in FIG. 1-2. The results of surface antigen analysis demonstrate that, in the cell population at passage 0 derived from Donor A in Comparative Example 1, the positive rates of MSC markers CD73 and CD90 were 98.5% and 83.6%, respectively, and the positive rate of epithelial cell marker CD326 was 74.6%. As shown in FIG. 1-2, in addition, a width of the histogram indicating CD90 expression is expanded. This indicates that CD90 expression levels are not uniform.

The above results demonstrate that a large quantity of epithelial cells are included in the cell population at passage 0 in Comparative Example 1 and such cell population comprises mesenchymal stem cells at low purity.

### Example 1: Examination of Amnion Storage

Process 1: Amnion Sampling and Storage

The amnion was sampled in the same manner as in Process 1 of Comparative Example 1, and the amnion cut into small pieces (about 1 g each) was accommodated in a 15-ml centrifuge tube containing 4 mL of a Hanks' balanced salt solution (without Ca and Mg). Thereafter, storage was carried out under refrigeration (4° C.) for 120 hours (5 days) or 216 hours (9 days), and Process 2 was then performed.

Process 2: Enzymatic Treatment of Amnion and Acquisition of Amnion-derived MSCs

From the amnion after storage described above, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

Concerning the cell population at passage 0 cultured in “Process 3: Culture of amnion-derived MSCs,” cell morphology was observed under an inverted culture microscope at any timing. The results are shown in FIG. 2-1.

The results demonstrate that epithelial cells are not included in the cell population at passage 0 derived from Donor A of Example 1 and that amnion-derived MSCs are proliferated over the entire culture surface.

Process 5: Surface Antigen Analysis

In the same manner as in Process 5 of Comparative Example 1, the cell population at passage 0 derived from Donor A in Example 1 was subjected to surface antigen analysis (the CD73 positive rate, the CD326 positive rate, and the CD326 positive rate) using a flow cytometer.

The results of surface antigen analysis are shown in FIG. 2-2. The results of surface antigen analysis are as follows. In the cell population at passage 0 obtained from the amnion derived from Donor A in Example 1 and stored for 120 hours, the positive rates of MSC markers CD73 and CD90 were 99.7% and 99.9%, respectively, and the positive rate of epithelial cell marker CD326 was 1.2% (the negative rate was 98.8%). In the cell population at passage 0 obtained from the amnion derived from Donor A in Example 1 and stored for 216 hours, the positive rates of MSC markers CD73 and CD90 were 98.8% and 99.6%, respectively, and the positive rate of epithelial cell marker CD326 was 0.3% (the negative rate was 99.7%). In the cell population at passage 0 obtained from the amnion derived from Donor A in Example 1 and stored for 120 or 216 hours, the CD326 positive rate was significantly lower than that of the cell population at passage 0 derived from the same Donor A in Comparative Example 1, and CD90 expression was uniform (i.e., the histogram is sharp).

The above results demonstrated that epithelial cells were not observed in the cell population at passage 0 derived from Donor A in Example 1 and that a cell population comprising mesenchymal stem cells at high purity could be produced.

### Example 2: Examination of Amnion Storage

Process 1: Amnion Sampling and Storage

The amnion was sampled in the same manner as in Process 1 of Comparative Example 1, and the amnion cut into small pieces (about 1 g each) was embedded in a 15-mL centrifuge tube containing 4 mL of a Hanks' balanced salt solution (without Ca and Mg) comprising 5% (w/v) gelatin. The amnion was embedded by liquefying the prepared Hanks' balanced salt solution comprising 5% (w/v) gelatin at 37° C., introducing the amnion into the solution, and cooling down to 4° C. immediately thereafter to for gelling the solution. Thereafter, storage was carried out under refrigeration (4° C.) for 120 hours (5 days) or 216 hours (9 days) and then heated to 37° C. to liquefy the Hanks' balanced salt solution comprising 5% (w/v) gelatin, and the amnion was isolated. Process 2 was then performed.

Process 2: Enzymatic Treatment of Amnion and Acquisition of Amnion-derived MSCs

From the amnion after storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

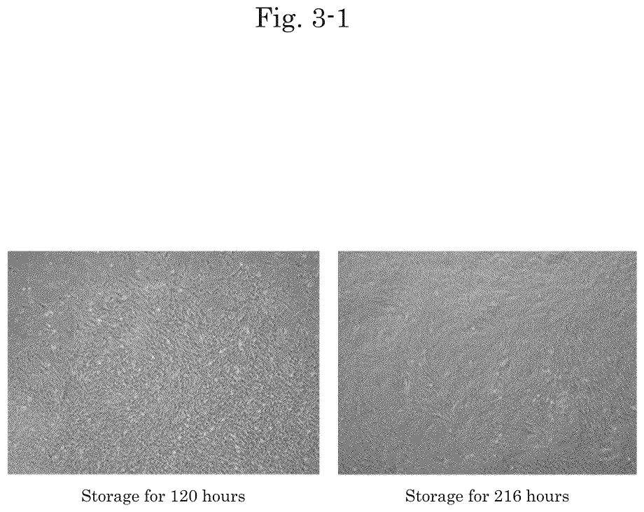

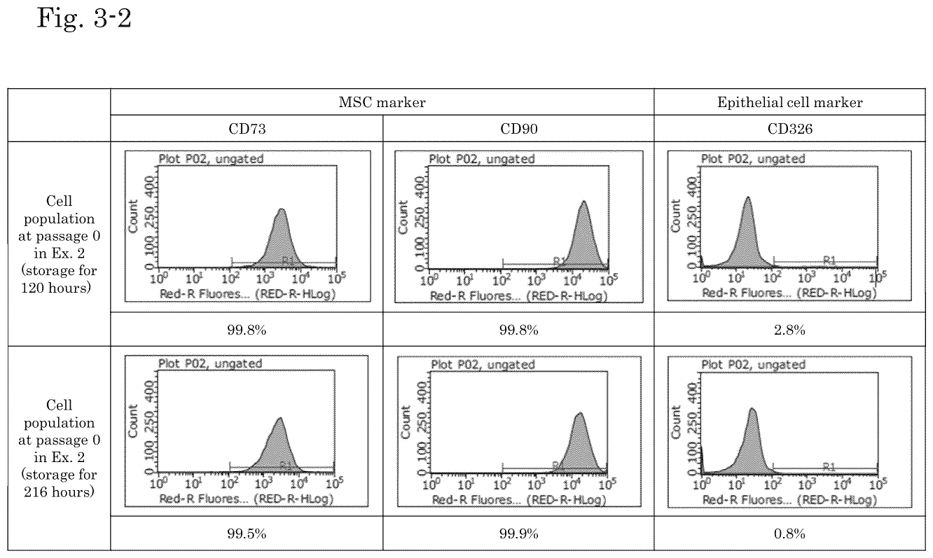

Concerning the cell population at passage 0 cultured in “Process 3: Culture of amnion-derived MSCs,” cell morphology was observed under an inverted culture microscope at any timing. The results are shown in FIG. 3-1.

The results demonstrate that inclusion of epithelial cells are not observed in the cell population at passage 0 derived from Donor A in Example 2 and that amnion-derived MSCs were proliferated over the entire culture surface.

Process 5: Surface Antigen Analysis

In the same manner as in Process 5 of Comparative Example 1, the cell population at passage 0 derived from Donor A in Example 2 was subjected to surface antigen analysis (the CD73 positive rate, the CD326 positive rate, and the CD326 positive rate) using a flow cytometer.

The results of surface antigen analysis are shown in FIG. 3-2. The results of surface antigen analysis are as follows. In the cell population at passage 0 obtained from the amnion derived from Donor A in Example 2 and stored for 120 hours, the positive rates of MSC markers CD73 and CD90 were 99.8% and 99.8%, respectively, and the positive rate of epithelial cell marker CD326 was 2.8% (the negative rate was 97.2%). In the cell population at passage 0 obtained from the amnion derived from Donor A in Example 2 and stored for 216 hours, the positive rates of MSC markers CD73 and CD90 were 99.5% and 99.9%, respectively, and the positive rate of epithelial cell marker CD326 was 0.8% (the negative rate was 99.2%). In the cell population at passage 0 obtained from the amnion derived from Donor A in Example 2 and stored for 120 or 216 hours, as with the case in Example 1, the CD326 positive rate was significantly lower than that of the cell population at passage 0 derived from the same Donor A in Comparative Example 1, and CD90 expression was uniform (i.e., the histogram is sharp).

The above results demonstrate that epithelial cells were not observed in the cell population at passage 0 in Example 2 and that a cell population comprising mesenchymal stem cells at high purity was produced.

In addition, the results of surface antigen analyses of the cell population at passage 0 performed in Example 1 and Example 2 demonstrated that the positive rate of an epithelial cell marker tends to be lower and the purity tends to be higher as the duration of amnion storage in the medium is longer.

### Example 3: Examination of Amnion Storage

Process 1: Amnion Sampling

From a pregnant woman of the elective cesarean section case from which the written informed consent was obtained (Donor B), the fetal membrane and the placenta which are fetal appendages were sampled aseptically. The fetal membrane and the placenta obtained were accommodated in a vessel containing physiological saline, and the amnion was isolated from the stump end of the fetal membrane. The amnion was washed using a Hanks' balanced salt solution (without Ca and Mg) and cut into small pieces. The amnion cut into small pieces (about 13 g) was accommodated in a square medium bottle containing 150 mL of a Hanks' balanced salt solution (without Ca and Mg), stored under refrigeration (4° C.) for 72 hours (3 days), and Process 2 was then performed.

Process 2: Enzymatic Treatment of Amnion and Acquisition of Amnion-derived MSCs

The amnion after the above storage (13 g) was agitated with shaking in a Hanks' balanced salt solution (with Ca and Mg) comprising 240 PU/mL collagenase and 200 PU/mL dispase I at 37° C. for 90 minutes and the amnion was thus subjected to enzymatic treatment. After the enzymatic treatment, the solution was filtered through a strainer to remove an undigested amnion fraction, and a cell fraction comprising amnion-derived MSCs was obtained.

Process 3: Culture of Amnion-derived MSCs

The cell fraction comprising amnion-derived MSCs obtained in “Process 2: Enzymatic treatment of amnion and acquisition of amnion-derived MSCs” above was seeded in a culture vessel (T-25 flask, Corning) at 1,000 cells/cm² and subjected to adherent culture in MEM-α (Minimum essential medium α) comprising 5% by volume (final concentration) of hPL. The cells subjected to adherent culture are referred to as the cell population at passage 0. When the cells reached a subconfluent state, the cells were detached with the use of TrypLE Select, diluted using physiological saline comprising 8% by volume of hPL, and collected by centrifugation. The collected cell population was suspended in a cryopreservation solution containing CP-1® (Kyokuto Pharmaceutical Industrial Co., Ltd.), 25% by mass human serum albumin, and physiological saline mixed at the proportion of 2:1:3, the cell suspension was slowly frozen to −80° C., and was thereafter cryopreserved at −80° C.

Process 4: Microscopic Observation of Cell Population at Passage 0 Comprising Cultured Amnion-derived MSCs

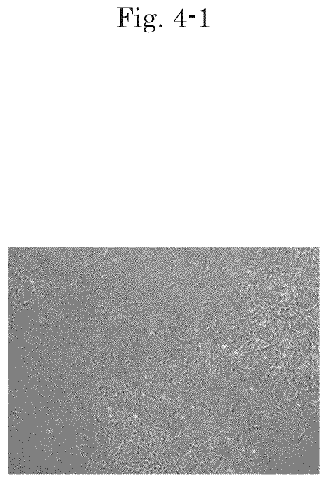

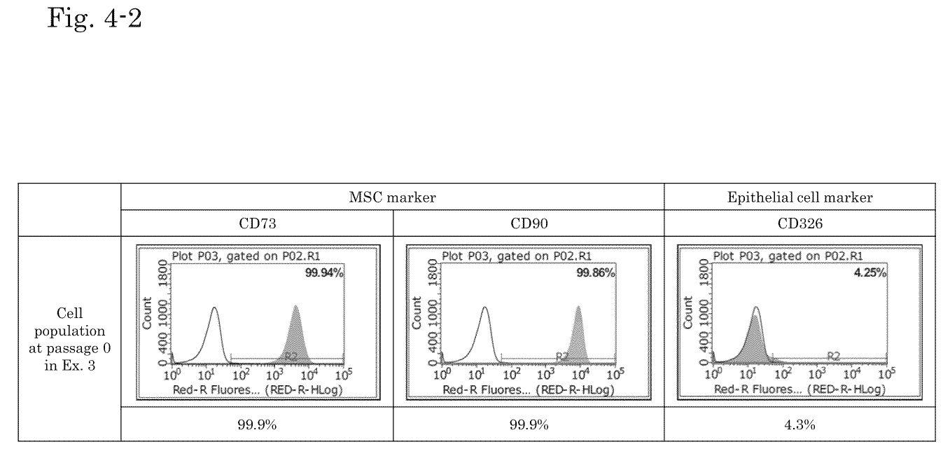

Concerning the cell population at passage 0 cultured in “Process 3: Culture of amnion-derived MSCs,” cell morphology was observed under an inverted culture microscope at any timing. The results are shown in FIG. 4-1.

The results demonstrate that inclusion of epithelial cells were not observed in the cell population at passage 0 derived from Donor B in Example 3 and that amnion-derived MSCs were proliferated.

Process 5: Surface Antigen Analysis

In the same manner as in Process 5 of Comparative Example 1, the cell population at passage 0 derived from Donor A in Example 3 was subjected to surface antigen analysis (the CD73 positive rate, the CD326 positive rate, and the CD326 positive rate) using a flow cytometer.

The results of surface antigen analysis are shown in FIG. 4-2. The results of surface antigen analysis are as follows. In the cell population at passage 0 derived from Donor B in Example 3, the positive rates of MSC markers CD73 and CD90 were 99.9% and 99.9%, respectively, and the positive rate of epithelial cell marker CD326 was 4.3%. In the cell population at passage 0 obtained from the amnion derived from Donor B in Example 3 and stored for 72 hours, the positive rate of CD326 was significantly low as in the case of the cell population at passage 0 obtained from the amnion derived from Donor A in Example 1 or Example 2 and stored for 120 hours or longer, and CD90 expression was uniform (i.e., the histogram is sharp).

The above results demonstrated that epithelial cells were not observed in the cell population at passage 0 of Example 3 and that a cell population comprising mesenchymal stem cells at high purity is produced.

### Example 4: Examination of Amnion Storage

Process 1: Amnion Sampling

The amnion cut into small pieces (about 1 g) treated in the same manner as in Comparative Example 1, Example 1, and Example 2 was accommodated in a 15-mL centrifuge tube containing 4 mL of MEM-α comprising 5% by volume of hPL. Thereafter, storage was carried out under refrigeration (4° C.) for 216 hours (9 days), and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

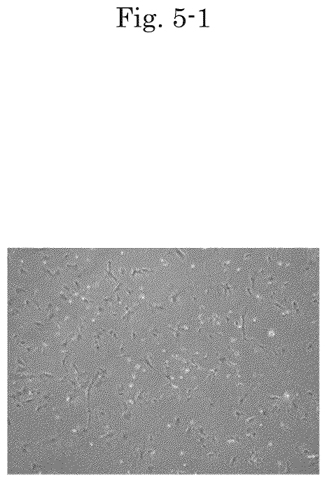

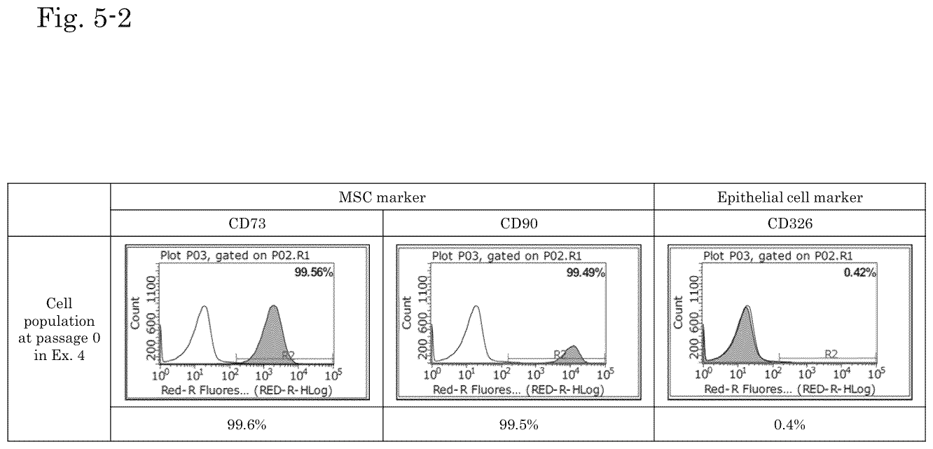

Concerning the cell population at passage 0 cultured in “Process 3: Culture of amnion-derived MSCs” described above, cell morphology was observed under an inverted culture microscope at any timing. The results are shown in FIG. 5-1.

The results demonstrate that inclusion of epithelial cells were not observed in the cell population at passage 0 derived from Donor A in Example 4 and that amnion-derived MSCs were proliferated over the entire culture surface.

Process 5: Surface Antigen Analysis

In the same manner as in Process 5 of Comparative Example 1, the cell population at passage 0 derived from Donor A in Example 4 was subjected to surface antigen analysis (the CD73 positive rate, the CD326 positive rate, and the CD326 positive rate) using a flow cytometer.

The results of surface antigen analysis are shown in FIG. 5-2. The results of surface antigen analysis are as follows. In the cell population at passage 0 derived from Donor A in Example 4, the positive rates of MSC markers CD73 and CD90 were 99.6% and 99.5%, respectively, and the positive rate of epithelial cell marker CD326 was 0.4% (the negative rate was 99.6%). In the cell population at passage 0 obtained from the amnion derived from Donor A in Example 4 and stored for 216 hours, the CD326 positive rate was significantly lower than that of the cell population at passage 0 derived from Donor A in Comparative Example 1, and CD90 expression was uniform (i.e., the histogram is sharp).

The above results demonstrated that epithelial cells were not observed in the cell population at passage 0 of Example 4 and that a cell population comprising mesenchymal stem cells at high purity could be produced.

### Example 5: Examination of Amnion Storage

Process 1: Amnion Sampling

From a pregnant woman of the elective cesarean section case from which the written informed consent was obtained (Donor C), the fetal membrane and the placenta which are fetal appendages were sampled aseptically. The fetal membrane and the placenta obtained were accommodated in a vessel containing physiological saline, and the amnion was isolated from the stump end of the fetal membrane. The amnion was washed using a Hanks' balanced salt solution (without Ca and Mg), and a washed amnion (about 40 g) was directly accommodated in a square medium bottle containing 500 mL of a Hanks' balanced salt solution (without Ca and Mg). Thereafter, storage was carried out under refrigeration (4° C.) for 5 hours, and Process 2 was then performed.

Process 2: Enzymatic Treatment of Amnion and Acquisition of Amnion-derived MSCs

The amnion after the above storage was isolated from the medium and subjected to enzymatic treatment and filtration in the same manner as in Process 2 of Comparative Example 1 to obtain a cell fraction comprising amnion-derived MSCs.

Process 3: Culture of Amnion-derived MSCs

The cell fraction comprising amnion-derived MSCs obtained in “Process 2: Enzymatic treatment of amnion and acquisition of amnion-derived MSCs” described above was seeded in a culture vessel (10-layer cell stack, Corning) at 1,000 cells/cm² and subjected to adherent culture in MEM-α (Minimum essential medium α) comprising 5% by volume (final concentration) of a human platelet lysate. The cells subjected to adherent culture are referred to as the cell population at passage 0. When the cells reached a subconfluent state, the cells were detached with the use of TrypLE Select, diluted using a medium, and collected by centrifugation. The collected cell population was suspended in a cryopreservation solution containing CP-1® (Kyokuto Pharmaceutical Industrial Co., Ltd.), 25% by mass human serum albumin, and physiological saline mixed at the proportion of 2:1:3, the cell suspension was slowly frozen to −80° C., and was thereafter cryopreserved at −80° C.

Process 4: Microscopic Observation of Cell Population at Passage 0 Comprising Cultured Amnion-derived MSCs

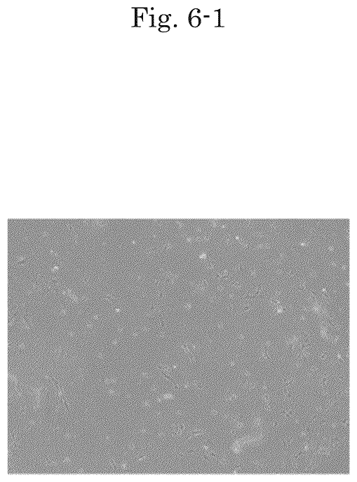

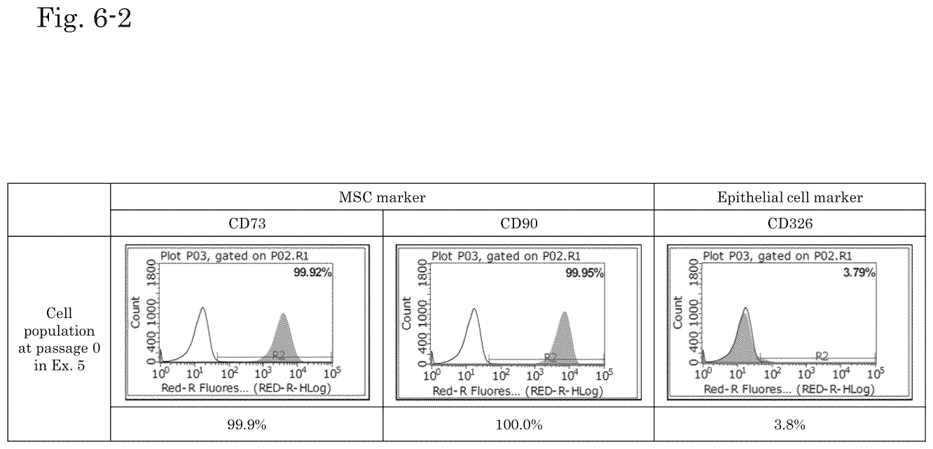

Concerning the cell population at passage 0 cultured in “Process 3: Culture of amnion-derived MSCs” described above, cell morphology was observed under an inverted culture microscope at any timing. The results are shown in FIG. 6-1.

The results demonstrate that inclusion of epithelial cells were not observed in the cell population at passage 0 derived from Donor C in Example 5 and that amnion-derived MSCs were proliferated over the entire culture surface.

Process 5: Surface Antigen Analysis

In the same manner as in Process 5 of Comparative Example 1, the cell population at passage 0 derived from Donor C of Example 5 was subjected to surface antigen analysis (the CD73 positive rate, the CD326 positive rate, and the CD326 positive rate) using a flow cytometer.

The results of surface antigen analysis are shown in FIG. 6-2. The results of surface antigen analysis are as follows. In the cell population at passage 0 derived from Donor C in Example 5, the positive rates of MSC markers CD73 and CD90 were 99.9% and 100.0%, respectively, and the positive rate of epithelial cell marker CD326 was 3.8%. In the cell population at passage 0 obtained from the amnion derived from Donor C in Example 5 and stored for 5 hours, the CD326 positive rate was significantly lower than that of the cell population at passage 0 derived from Donor A in Comparative Example 1, and CD90 expression was uniform (i.e., the histogram is sharp).

The above results demonstrated that epithelial cells were not observed in the cell population at passage 0 of Example 5 and that a cell population comprising mesenchymal stem cells at high purity could be produced.

From the above results, a method in the examples satisfying the following conditions (1), (2), and (3) was found to be capable of producing high-purity amnion-derived MSCs:
(1) the amnion is stored in a medium for 4 hours or longer at −1° C. or higher and 10° C. or lower;
(2) thereafter, the amnion is subjected to enzymatic treatment; and
(3) following the enzymatic treatment, a cell fraction comprising mesenchymal stem cells is cultured.

Thus, according to the present invention, a cell population comprising mesenchymal stem cells at high purity can be efficiently isolated from the amnion. Accordingly, expanded opportunities for treatment provided to patients, a reduced burden on those who perform cell culture, and a reduction in the production cost or medical expenses can be expected.

### Example 6: Examination of Amnion Storage

Process 1: Amnion Sampling and Storage

In the same manner as in Process 1 of Comparative Example 1, the amnion was sampled from a donor (Donor B) different from the donors in Comparative Example 1 and Examples 1 to 5. The amnion cut into small pieces (about 1 g each) was accommodated in a 15-mL centrifuge tube containing 4 mL of a Hanks' balanced salt solution (without Ca and Mg). Thereafter, storage was carried out under refrigeration (10° C.) for 48 hours (2 days) or 144 hours (6 days), respectively, and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

Concerning the cell population at passage 0 cultured in “Process 3: Culture of amnion-derived MSCs” described above, cell morphology was observed under an inverted culture microscope at any timing.

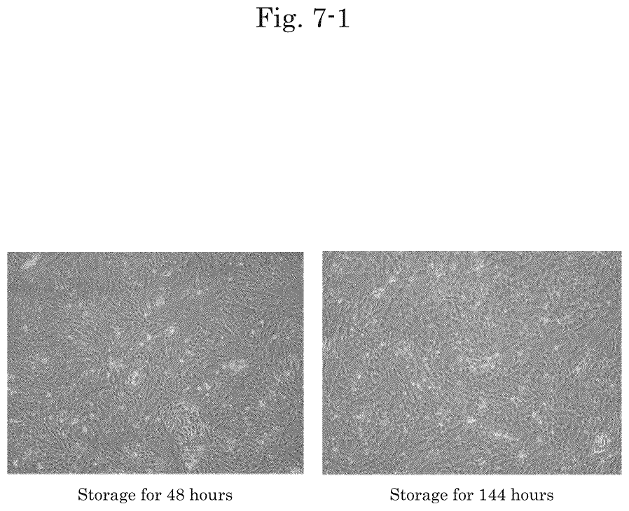

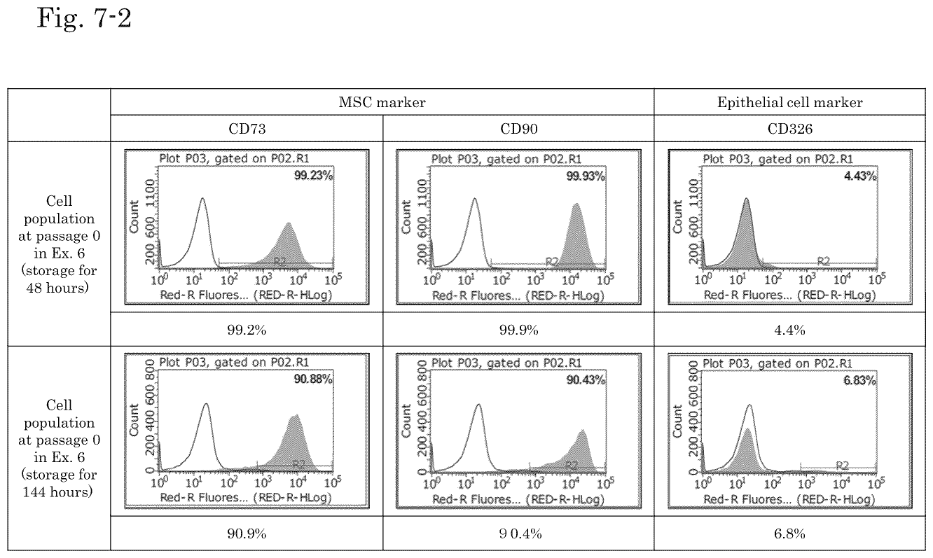

The results are shown in FIG. 7-1. The results demonstrate that inclusion of epithelial cells were not observed in the cell population at passage 0 derived from Donor B in Example 6 and that amnion-derived MSCs were proliferated over the entire culture surface (FIG. 7-1).

Process 5: Surface Antigen Analysis

In the same manner as in Process 5 of Comparative Example 1, the cell population at passage 0 derived from Donor B in Example 6 was subjected to surface antigen analysis (the CD73 positive rate, the CD326 positive rate, and the CD326 positive rate) using a flow cytometer.

The results of surface antigen analysis are shown in FIG. 7-2. In the cell population at passage 0 obtained from the amnion derived from Donor B in Example 6 and stored for 48 hours, the positive rates of MSC markers CD73 and CD90 were 99.2% and 99.9%, respectively, and the positive rate of epithelial cell marker CD326 was 4.4% (the negative rate was 95.6%). In the cell population at passage 0 obtained from the amnion derived from Donor B in Example 6 and stored for 144 hours, the positive rates of MSC markers CD73 and CD90 were 90.9% and 90.4%, respectively, and the positive rate of epithelial cell marker CD326 was 6.8% (the negative rate was 93.2%). In the cell population at passage 0 obtained from the amnion derived from Donor B in Example 6 and stored for 48 hours or 144 hours respectively, the CD326 positive rate was significantly lower than that of the cell population at passage 0 derived from Donor A in Comparative Example 1, and CD90 expression was uniform (i.e., the histogram is sharp).

The above results demonstrated that epithelial cells were not observed in the cell population at passage 0 derived from Donor B in Example 6 and that a cell population comprising mesenchymal stem cells at high purity could be produced.

### Example 7: Examination of Amnion Storage

Process 1: Amnion Sampling and Storage

In the same manner as in Process 1 of Comparative Example 1, the amnion was sampled from a donor (Donor B) different from the donors in Comparative Example 1 and Examples 1 to 5, and the amnion cut into small pieces (about 1 g each) was accommodated in a 15-mL centrifuge tube containing 4 mL of a Hanks' balanced salt solution (without Ca and Mg) comprising 5% (w/v) gelatin. Thereafter, storage was carried out under refrigeration (10° C.) for 48 hours (2 days) or 144 hours (6 days) respectively, and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

Concerning the cell population at passage 0 cultured in “Process 3: Culture of amnion-derived MSCs” described above, cell morphology was observed under an inverted culture microscope at any timing.

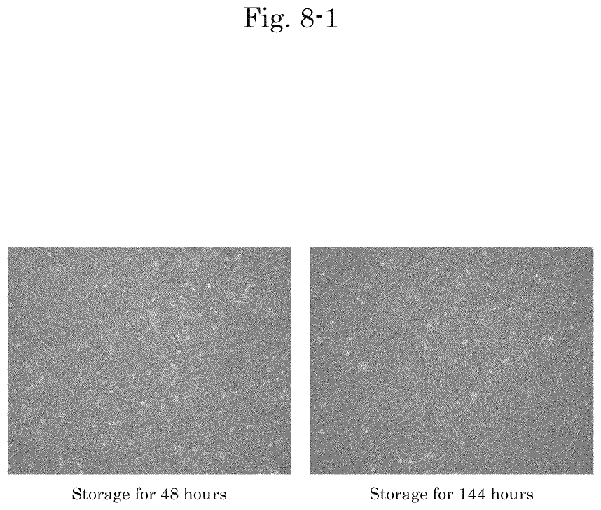

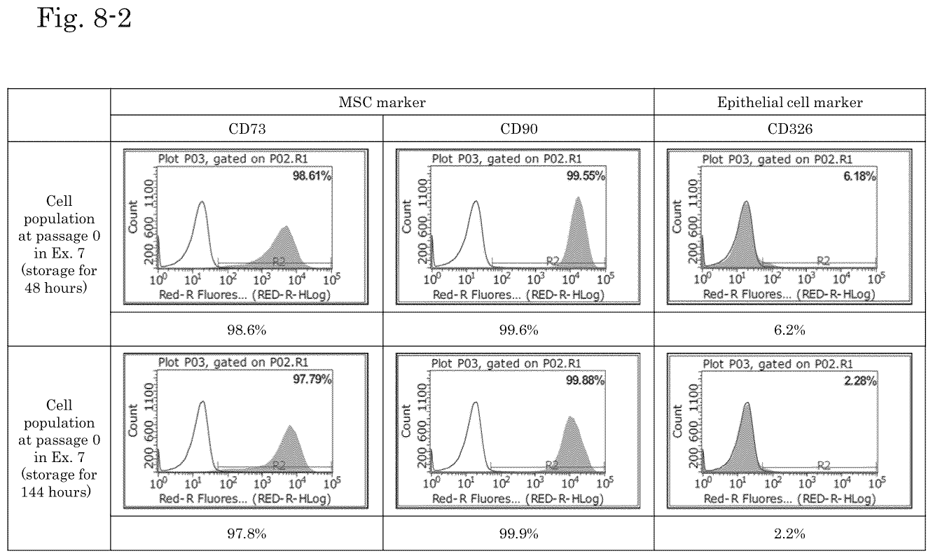

The results are shown in FIG. 8-1. The results demonstrate that inclusion of epithelial cells were not observed in the cell population at passage 0 derived from Donor B in Example 7 and that amnion-derived MSCs were proliferated over the entire culture surface (FIG. 8-1).

Process 5: Surface Antigen Analysis

In the same manner as in Process 5 of Comparative Example 1, the cell population at passage 0 derived from Donor B in Example 7 was subjected to surface antigen analysis (the CD73 positive rate, the CD326 positive rate, and the CD326 positive rate) using a flow cytometer.

The results of surface antigen analysis are shown in FIG. 8-2. In the cell population at passage 0 obtained from the amnion derived from Donor B in Example 7 and stored for 48 hours, the positive rates of MSC markers CD73 and CD90 were 98.6% and 99.6%, respectively, and the positive rate of epithelial cell marker CD326 was 6.2% (the negative rate was 93.8%). In the cell population at passage 0 obtained from the amnion derived from Donor B in Example 7 and stored for 144 hours, the positive rates of MSC markers CD73 and CD90 were 97.8% and 99.9%, respectively, and the positive rate of epithelial cell marker CD326 was 2.2% (the negative rate was 97.8%). In the cell population at passage 0 obtained from the amnion derived from Donor B in Example 7 and stored for 48 hours or 144 hours respectively, the CD326 positive rate was significantly lower than that of the cell population at passage 0 derived from Donor A in Comparative Example 1, and CD90 expression was uniform (i.e., the histogram is sharp).

The above results demonstrated that epithelial cells were not observed in the cell population at passage 0 derived from Donor B in Example 7 and that a cell population comprising mesenchymal stem cells at high purity could be produced.

### Example 8: Examination of Amnion Storage

Process 1: Amnion Sampling and Storage

In the same manner as in Process 1 of Comparative Example 1, the amnion was sampled from a donor (Donor B) different from the donors in Comparative Example 1 and Examples 1 to 5, and the amnion cut into small pieces (about 1 g each) was accommodated in a 15-mL centrifuge tube containing 4 mL of MEM-α comprising 5% by volume of hPL. Thereafter, storage was carried out under refrigeration (10° C.) for 48 hours (2 days) or 144 hours (6 days) respectively, and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

Concerning the cell population at passage 0 cultured in “Process 3: Culture of amnion-derived MSCs” described above, cell morphology was observed under an inverted culture microscope at any timing.

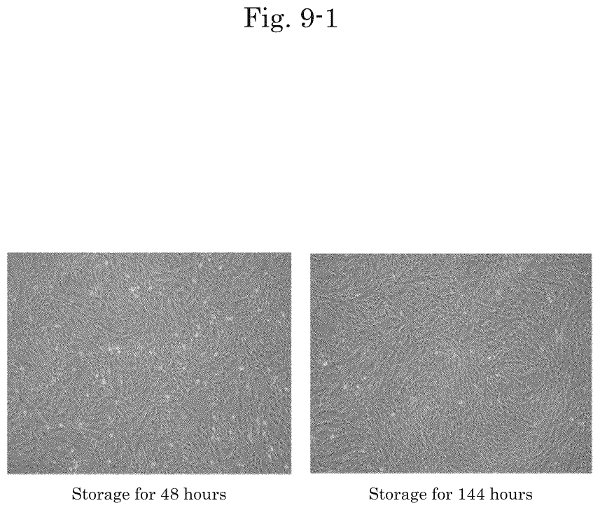

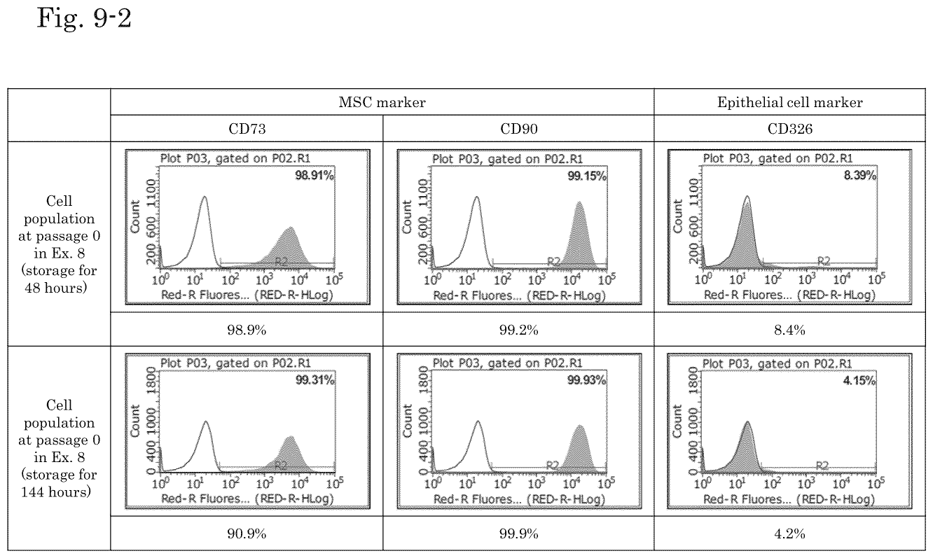

The results are shown in FIG. 9-1. The results demonstrate that inclusion of epithelial cells was not observed in the cell population at passage 0 derived from Donor B in Example 8 and that amnion-derived MSCs were proliferated over the entire culture surface (FIG. 9-1).

Process 5: Surface Antigen Analysis

In the same manner as in Process 5 of Comparative Example 1, the cell population at passage 0 derived from Donor B in Example 8 was subjected to surface antigen analysis (the CD73 positive rate, the CD326 positive rate, and the CD326 positive rate) using a flow cytometer.

The results of surface antigen analysis are shown in FIG. 9-2. In the cell population at passage 0 obtained from the amnion derived from Donor B in Example 8 and stored for 48 hours, the positive rates of MSC markers CD73 and CD90 were 98.9% and 99.2%, respectively, and the positive rate of epithelial cell marker CD326 was 8.4% (the negative rate was 91.6%). In the cell population at passage 0 obtained from the amnion derived from Donor B in Example 8 and stored for 144 hours, the positive rates of MSC markers CD73 and CD90 were 90.9% and 99.9%, respectively, and the positive rate of epithelial cell marker CD326 was 4.2% (the negative rate was 95.8%). In the cell population at passage 0 obtained from the amnion derived from Donor B in Example 8 and stored for 48 hours or 144 hours respectively, the CD326 positive rate was significantly lower than that of the cell population at passage 0 derived from Donor A in Comparative Example 1, and CD90 expression was uniform (i.e., the histogram is sharp).

The above results demonstrated that epithelial cells were not observed in the cell population at passage 0 derived from Donor B in Example 8 and that a cell population comprising mesenchymal stem cells at high purity could be produced.

### Example 9: Examination of Amnion Storage

Process 1: Amnion Sampling and Storage

In the same manner as in Process 1 of Comparative Example 1, the amnion was sampled from a donor (Donor C) different from the donors in Comparative Example 1 and Examples 1 to 8, and cut amnion (about 10 g) was accommodated in a 250-mL square bottle containing 150 mL of a Hanks' balanced salt solution (without Ca and Mg). Thereafter, storage was carried out under refrigeration (4° C.) for 4 hours or 94 hours (4 days) respectively, and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

Concerning the cell population at passage 0 cultured in “Process 3: Culture of amnion-derived MSCs” described above, cell morphology was observed under an inverted culture microscope at any timing.

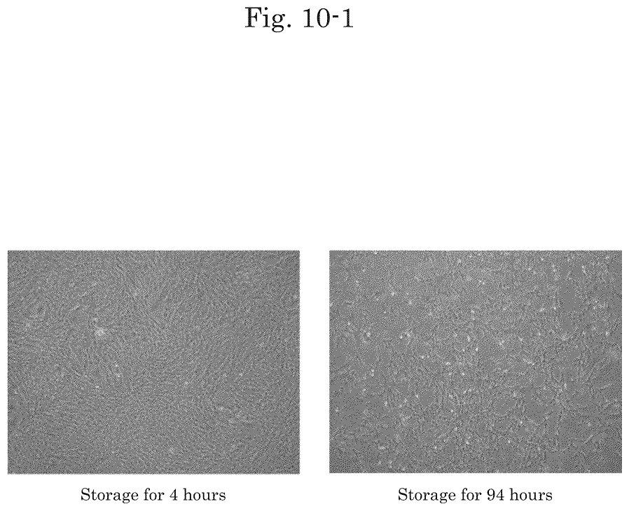

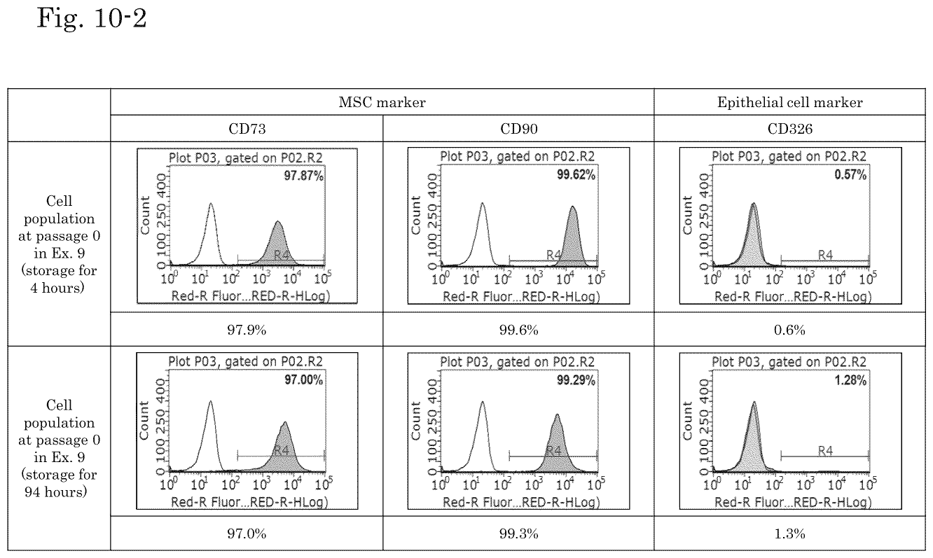

The results are shown in FIG. 10-1. The results demonstrated that inclusion of epithelial cells were not observed in the cell population at passage 0 derived from Donor B in Example 9 and that amnion-derived MSCs were proliferated over the entire culture surface (FIG. 10-1).

Process 5: Surface Antigen Analysis

In the same manner as in Process 5 of Comparative Example 1, the cell population at passage 0 derived from Donor C in Example 9 was subjected to surface antigen analysis (the CD73 positive rate, the CD326 positive rate, and the CD326 positive rate) using a flow cytometer.

The results of surface antigen analysis are shown in FIG. 10-2. In the cell population at passage 0 obtained from the amnion derived from Donor C in Example 9 and stored for 4 hours, the positive rates of MSC markers CD73 and CD90 were 97.9% and 99.6%, respectively, and the positive rate of epithelial cell marker CD326 was 0.6% (the negative rate was 99.4%). In the cell population at passage 0 obtained from the amnion derived from Donor C in Example 9 and stored for 94 hours, the positive rates of MSC markers CD73 and CD90 were 97.0% and 99.3%, respectively, and the positive rate of epithelial cell marker CD326 was 1.3% (the negative rate was 98.7%). In the cell population at passage 0 obtained from the amnion derived from Donor C in Example 9 and stored for 4 hours or 94 hours respectively, the CD326 positive rate was significantly lower than that of the cell population at passage 0 derived from Donor A in Comparative Example 1, and CD90 expression was uniform (i.e., the histogram is sharp).

The above results demonstrated that epithelial cells were not observed in the cell population at passage 0 derived from Donor C in Example 9 and that a cell population comprising mesenchymal stem cells at high purity could be produced.

### Example 10: Examination of Amnion Storage

Process 1: Amnion Sampling and Storage

In the same manner as in Process 1 of Comparative Example 1, the amnion was sampled from a donor (Donor C) different from the donors in Comparative Example 1 and Examples 1 to 8, and cut amnion (about 9 g) was accommodated in a 250-mL square bottle containing 150 mL of a Hanks' balanced salt solution (without Ca and Mg). Thereafter, storage was carried out at room temperature (20° C.) for 4 hours or 94 hours (4 days) respectively, and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

Concerning the cell population at passage 0 cultured in “Process 3: Culture of amnion-derived MSCs” described above, cell morphology was observed under an inverted culture microscope at any timing.

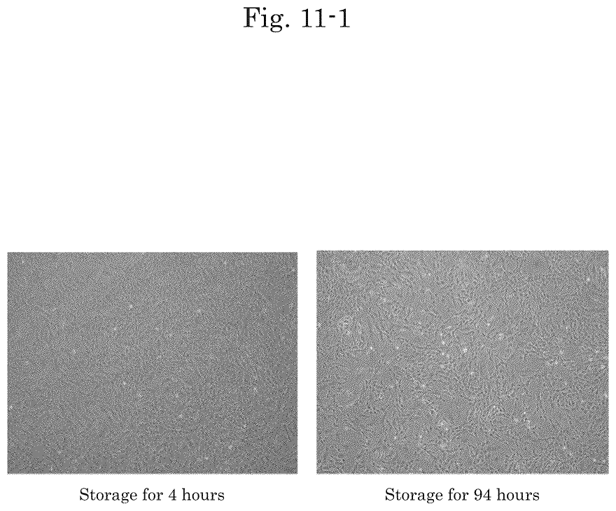

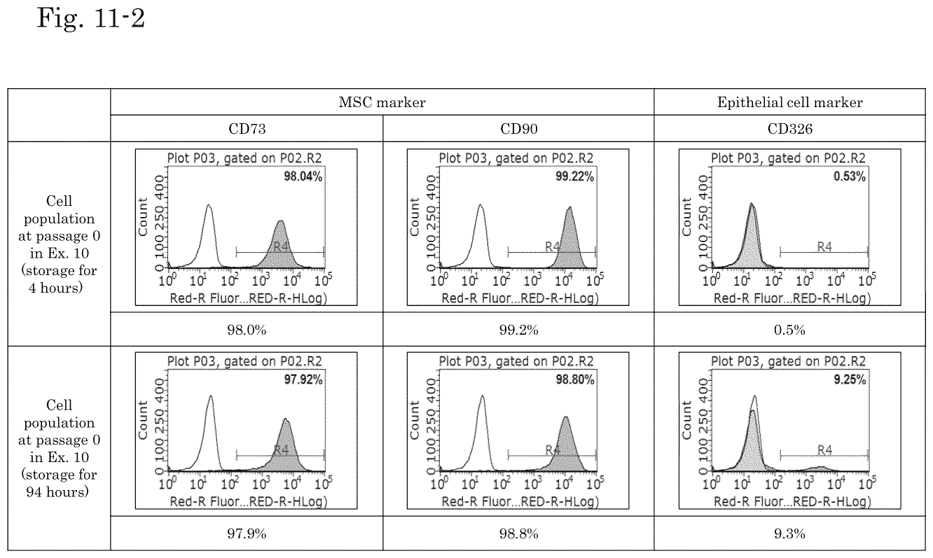

The results are shown in FIG. 11-1. The results demonstrated that inclusion of epithelial cells were not observed in the cell population at passage 0 derived from Donor C in Example 10 and that amnion-derived MSCs were proliferated over the entire culture surface (FIG. 11-1).

Process 5: Surface Antigen Analysis

In the same manner as in Process 5 of Comparative Example 1, the cell population at passage 0 derived from Donor C in Example 10 was subjected to surface antigen analysis (the CD73 positive rate, the CD326 positive rate, and the CD326 positive rate) using a flow cytometer.

The results of surface antigen analysis are shown in FIG. 11-2. In the cell population at passage 0 obtained from the amnion derived from Donor C in Example 10 and stored for 4 hours, the positive rates of MSC markers CD73 and CD90 were 98.0% and 99.2%, respectively, and the positive rate of epithelial cell marker CD326 was 0.5% (the negative rate was 99.5%). In the cell population at passage 0 obtained from the amnion derived from Donor C in Example 9 and stored for 94 hours, the positive rates of MSC markers CD73 and CD90 were 97.9% and 98.8%, respectively, and the positive rate of epithelial cell marker CD326 was 9.3% (the negative rate was 90.7%). In the cell population at passage 0 obtained from the amnion derived from Donor C in Example 10 and stored for 4 hours or 94 hours respectively, the CD326 positive rate was significantly lower than that of the cell population at passage 0 derived from Donor A in Comparative Example 1, and CD90 expression was uniform (i.e., the histogram is sharp).

The above results demonstrated that epithelial cells were not observed in the cell population at passage 0 derived from Donor C in Example 9 and that a cell population comprising mesenchymal stem cells at high purity could be produced.

### Comparative Example 2: Examination of Amnion Storage

Process 1: Amnion Sampling and Storage

In the same manner as in Process 1 of Comparative Example 1, the amnion was sampled from a donor (Donor C) different from the donors in Comparative Example 1 and Examples 1 to 8, and cut amnion (about 5 g) was accommodated in a 250-mL square bottle containing 150 mL of a Hanks' balanced salt solution (without Ca and Mg). Thereafter, storage was carried out at 37° C. for 4 hours, and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell population at passage 0 was obtained and collected from the cell fraction comprising amnion-derived MSCs of Process 2.

Process 4: Microscopic Observation of Cell Population at Passage 0 Comprising Cultured Amnion-derived MSCs

Concerning the cell population at passage 0 cultured in “Process 3: Culture of amnion-derived MSCs” described above, cell morphology was observed under an inverted culture microscope at any timing.

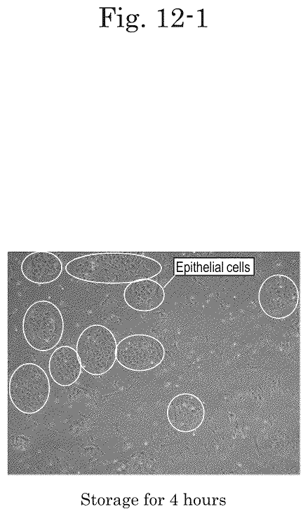

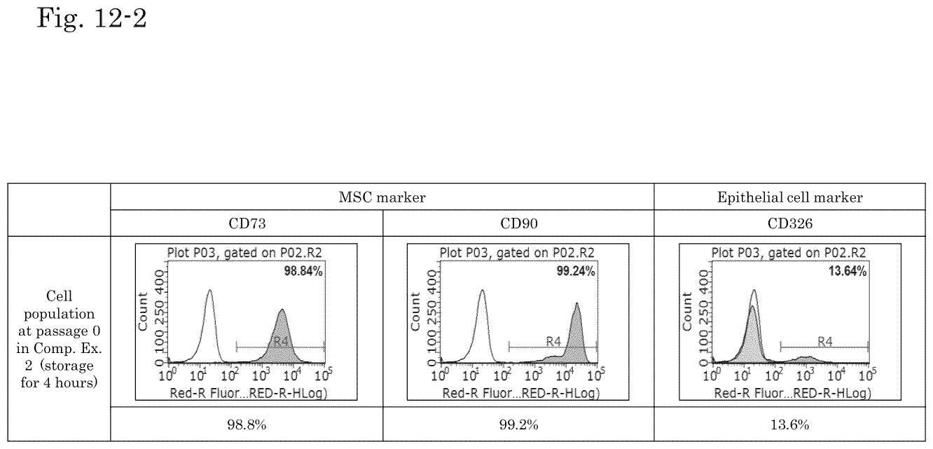

The results are shown in FIG. 12-1. The results demonstrated that, in addition to amnion-derived MSCs, a large number of epithelial cells (circles in FIG. 12-1) were included in the cell population at passage 0 derived from Donor C in Comparative Example 2 (FIG. 12-1).

Process 5: Surface Antigen Analysis

In the same manner as in Process 5 of Comparative Example 1, the cell population at passage 0 derived from Donor C in Comparative Example 2 was subjected to surface antigen analysis (the CD73 positive rate, the CD326 positive rate, and the CD326 positive rate) using a flow cytometer.

The results of surface antigen analysis are shown in FIG. 12-2. In the cell population at passage 0 obtained from the amnion derived from Donor C in Comparative Example 2 and stored for 4 hours, the positive rates of MSC markers CD73 and CD90 were 98.8% and 99.2%, respectively, and the positive rate of epithelial cell marker CD326 was 13.6%. As shown in Table 12, in addition, a width of the histogram indicating CD90 expression was expanded, indicating that CD90 expression levels were not uniform.

The above results demonstrated that many epithelial cells were included in the cell population at passage 0 derived from Donor C in Comparative Example 2 and that such cell population included mesenchymal stem cells at low purity.

### Comparative Example 3: Examination of Amnion Storage

Process 1: Amnion Sampling and Storage

In the same manner as in Process 1 of Comparative Example 1, the amnion was sampled from a donor (Donor C) different from the donors in Comparative Example 1 and Examples 1 to 8, and cut amnion (about 5 g) was accommodated in a 250-mL square bottle containing 150 mL of a Hanks' balanced salt solution (without Ca and Mg). Thereafter, storage was carried out at 37° C. for 94 hours, and Process 2 was then performed.

Process 2: Enzymatic Treatment of Amnion and Acquisition of Amnion-derived MSCs

From the amnion after the above storage, the cell fraction comprising amnion-derived MSCs was obtained in the same manner as in Process 2 of Comparative Example 1.

Process 3: Culture of Amnion-derived MSCs

In the same manner as in Process 3 of Comparative Example 1, the cell fraction comprising amnion-derived MSCs of Process 2 was cultured.

Process 4: Microscopic Observation of Cell Population at Passage 0 Comprising Cultured Amnion-derived MSCs

Concerning the cell population at passage 0 cultured in “Process 3: Culture of amnion-derived MSCs” described above, cell morphology was observed under an inverted culture microscope at any timing.

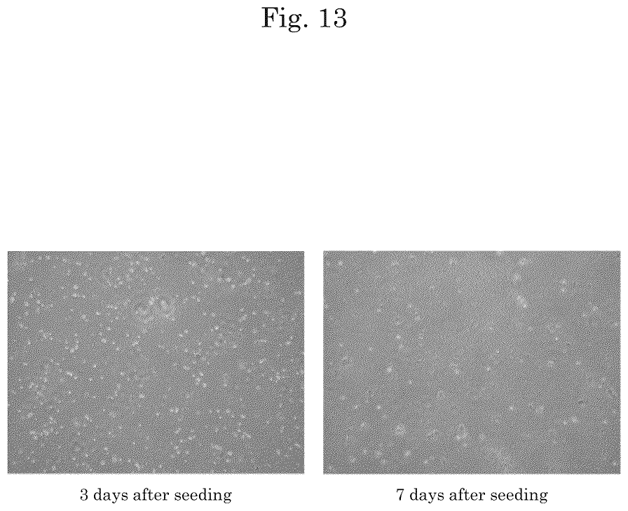

The results are shown in FIG. 13. The cell population at passage 0 derived from Donor C in Comparative Example 3 did not adhere to a culture vessel, and did not proliferate (FIG. 13).

The above results demonstrated that the cell population at passage 0 derived from Donor C in Comparative Example 3 does not show adhesion or proliferation.

### Effects of Amnion Storage Temperature on Mesenchymal Stem Cells and Epithelial Cells

The following Table 1 and Table 2 show the results of measurements of the amounts of cell collection in the examples and comparative examples described above.

TABLE 1
Amount of cell collection when amnion was stored for 4 hours

| | Storage temperature | | |
|---|---|---|---|
| | 4° C. | 20° C. | 37° C. |
| Viability (%) | 87.7 | 91.3 | 90.3 |
| Cell number per g of amnion | 4.02 × 10⁶ | 3.17 × 10⁶ | 4.50 × 10⁶ |

TABLE 2
Amount of cell collection when amnion was stored for 94 hours

| | Storage temperature | | |
|---|---|---|---|
| | 4° C. | 20° C. | 37° C. |
| Viability (%) | 64.7 | 84.7 | 77.0 |
| Cell number per g of amnion | 2.18 × 10⁶ | 1.67 × 10⁶ | 1.87 × 10⁷ |

The results shown in Table 1 and Table 2 demonstrated that there were no significant differences detected between different storage temperatures in respect of the cell viability and the cell number per g of the amnion for the overall cells including both mesenchymal stem cells and epithelial cells.

The above results demonstrated that, in the cell population obtained from the amnion stored at 37° C., the viability or the number of the entire cells including mesenchymal stem cells was not decreased, and that the proportion of the mesenchymal stem cells relative to the entire cells was decreased since epithelial cells selectively remain alive. Accordingly, it was found that a cell population comprising mesenchymal stem cells at a high rate cannot be obtained if the amnion is stored at 37° C.

In contrast, it was found that in the cell population obtained from the amnion stored at 4° C. or 20° C., epithelial cells selectively die. Accordingly, by performing amnion storage at low temperature of about 25° C. or lower, the proportion of epithelial cells can be selectively lowered, and the resulting cell population can comprise mesenchymal stem cells at a high rate, providing advantageous effects.

All publications, patents, and patent applications cited herein are incorporated herein by reference in their entirety.

1. A method of producing a cell population comprising amnion-derived mesenchymal stem cells, comprising
(1) a step of storing an amnion in a medium for 4 hours or longer at −1° C. or higher and 25° C. or lower;
(2) a step of treating the amnion with one or more enzymes thereafter, to obtain an enzyme treated solution;
(3) a step of removing an undigested amnion fraction from said enzyme treated solution to obtain an enzyme digested fraction having a reduced proportion of epithelial cells and comprising amnion-derived mesenchymal stem cells; and
(4) a step of culturing said enzyme digested amnion fraction comprising mesenchymal stem cells after the treatment with the one or more enzymes.

2. The method of producing a cell population comprising mesenchymal stem cells according to claim 1, further comprising isolating the amnion from the medium before the treatment with one or more enzymes.

3. The method of producing a cell population comprising mesenchymal stem cells according to claim 1, wherein the medium is an aqueous solution, a gel, or a sol.

4. The method of producing a cell population comprising mesenchymal stem cells according to claim 1, wherein the one or more enzymes comprise at least one selected from the group consisting of trypsin, collagenase, and dispase.

5. The method of producing a cell population comprising mesenchymal stem cells according to claim 1, wherein the amnion stored in the medium in the step (1) is shredded.

6. The method of producing a cell population comprising mesenchymal stem cells according to claim 1, wherein the amnion is stored at −1° C. or higher and 10° C. or lower in the step (1).
